(19) United States
(12) Patent Application Publication
(10) Pub. No.: US 2026/0123766 A1
(43) Pub. Date: May 7, 2026

(54) MULTI-FUNCTIONAL SUPPORT SYSTEM FOR SIMULATING BUOYANCY USING WEIGHTED MODULES

(71) Applicant: Kaniwar Wasi Ali, Hendersonville, TN (US)

(72) Inventor: Kaniwar Wasi Ali, Hendersonville, TN (US)

(21) Appl. No.: 19/336,592

(22) Filed: Sep. 23, 2025

Related U.S. Application Data
(60) Provisional application No. 63/717,164, filed on Nov. 6, 2024.

(51) Int. Cl. A47C 27/10; A47C 27/08; A47C 31/00

(52) U.S. Cl. CPC A47C 27/10; A47C 27/083; A47C 27/084; A47C 27/085; A47C 27/086; A47C 31/002; A47C 31/007; A47C 31/008

(57) ABSTRACT

The present invention relates to a multi-functional support system employing weighted modules configured to simulate buoyancy on land, thereby providing enhanced comfort, pressure distribution, and floating sensations for applications such as sleep, therapy, recreation, and ergonomic work environments. The multi-functional support system comprises a supporting frame, a plurality of weighted modules, and a ventilation unit. The multi-functional support system avoids the drawbacks of water-based flotation devices, such as leakage, heavy weight, and high maintenance requirements, while still delivering the therapeutic benefits of buoyant support. The multi-functional support system is adaptable in size, density, and configuration to meet the needs of diverse users, including children, adults, and individuals with limited mobility, and that incorporates features for breathability, hygiene, safety, and long-term durability.

### FIELD OF THE INVENTION

The present invention relates to ergonomic and therapeutic support systems, and more particularly to a multi-functional support system employing weighted modules configured to simulate buoyancy on land, thereby providing enhanced comfort, pressure distribution, and floating sensations for applications such as sleep, therapy, recreation, and ergonomic work environments.

### BACKGROUND

Conventional sleeping and seating arrangements, including mattresses, cushions, and chairs, are commonly employed to provide support and rest for the human body. However, these traditional systems typically utilize firm or semi-firm surfaces that generate localized pressure points at areas of body contact. The formation of such pressure points can impede blood circulation, induce discomfort during prolonged use, and contribute to adverse conditions such as muscle stiffness, back pain, and disturbed sleep.

To overcome these limitations, specialized products such as memory foam mattresses, spring-based beds, and ergonomic seating systems have been introduced. These designs aim to redistribute body weight and reduce discomfort by providing variable support across different regions of the body. Although such systems offer certain improvements, they still fall short of replicating the uniform weight distribution and pressure relief naturally achieved in buoyant water environments.

Water-based systems, such as flotation tanks and waterbeds, represent another category of prior art developed to alleviate pressure points through the principle of buoyancy. In flotation tanks, the user is immersed in a highly concentrated saltwater solution, which creates near-weightless conditions that substantially reduce mechanical loading on muscles and joints, thereby promoting deep relaxation. Similarly, waterbeds utilize a fluid-filled bladder that dynamically conforms to the body's contours, distributing weight more evenly compared to conventional solid or semi-solid surfaces. However, such systems may suffer from drawbacks including high maintenance requirements, risk of leakage, thermal regulation challenges, and limited adaptability to different user needs.

Further, the conventional water-based systems are associated with several inherent drawbacks that limit their widespread adoption. Flotation tanks, for instance, necessitate specialized infrastructure, occupy considerable space, and require large volumes of water along with continuous maintenance involving filtration, sanitation, and chemical treatment. These factors render them costly and impractical for routine household use. Waterbeds, although relatively more accessible, introduce additional challenges, such as excessive weight that complicates installation and relocation, susceptibility to leakage and puncture, and difficulties in achieving consistent thermal regulation of the fluid medium. Furthermore, both flotation tanks and waterbeds often lack portability, adaptability to different environments, and long-term durability. Consequently, their use remains largely confined to niche applications rather than becoming a practical solution for mainstream comfort and support needs.

Certain prior art solutions have attempted to replicate the buoyant characteristics of water without employing liquid-based systems, including air-filled cushions, inflatable loungers, and ball-pit style recreational products. While these designs are generally lighter, more portable, and easier to maintain compared to water-based systems, they lack the density and uniform resistance necessary to simulate the authentic floating sensation of water. Conventional air-filled structures, in particular, tend to compress excessively under body weight, leading to uneven pressure distribution, localized strain, and discomfort during extended use.

Furthermore, air-based or other lightweight designs frequently suffer from inadequate breathability, compromised hygiene, and limited long-term durability. Such systems are prone to trapping heat, fostering bacterial growth, and deteriorating under prolonged or repeated use. Recreational ball-pit configurations, while superficially similar in concept, are neither designed for ergonomic nor therapeutic applications and present safety concerns due to their instability and poor weight-distribution characteristics. Consequently, the prior art has yet to provide a solution that successfully integrates buoyant-like comfort with practical durability, hygiene, and versatility across diverse applications.

To address these limitations, there is a need for a multi-functional support system employing weighted modules configured to simulate buoyancy on land, thereby providing enhanced comfort, pressure distribution, and floating sensations for applications such as sleep, therapy, recreation, and ergonomic work environments. There is also a need for a multi-functional support system that avoids the drawbacks of water-based flotation devices, such as leakage, heavy weight, and high maintenance requirements, while still delivering the therapeutic benefits of buoyant support for users. Further, there is also a need for a multi-functional support system that is adaptable in size, density, and configuration to meet the needs of diverse users, including children, adults, and individuals with limited mobility, and that incorporates features for breathability, hygiene, safety, and long-term durability.

### SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key nor critical elements of all embodiments, nor delineate the scope of any or all embodiments.

The present invention provides a multi-functional support system configured to simulate the sensation of buoyancy experienced in aquatic environments while enabling use on land. The multi-functional support system comprises a supporting frame having sidewalls and a base defining a containment region that houses a plurality of weighted modules. Each of the plurality of weighted modules includes an outer shell enclosing an inner core, the density of which is calibrated to approximate human body density. The plurality of weighted modules is movable relative to the adjacent weighted modules within the containment region by a displacement under a static load, thereby redistributing beneath a user's body to provide buoyant support and reduce localized pressure points.

In one aspect, the multi-functional support system addresses longstanding problems associated with conventional beds, chairs, and therapeutic supports, which often concentrate pressure at contact points and contribute to discomfort, circulatory restriction, and musculoskeletal stress. By contrast, the multi-functional support system emulates the buoyant characteristics of water, thereby reducing localized pressure, promoting improved circulation, and enhancing overall user comfort. The multi-functional support system is suitable for sleeping, therapeutic rehabilitation, recreational use, and ergonomic workspace applications.

In some embodiments, the inner core of the plurality of weighted modules may comprise at least one of, but not limited to, a liquid, a metallic material, and a magnetic material selected to achieve a target density relative to the human body. The outer shell may be fabricated from at least one of a transparent polymer and a semi-transparent composite material. The outer shell comprises a shape includes at least one of, but not limited to, a spherical, an ellipsoidal, and a polyhedral. The outer shell may further incorporate at least one of, but not limited to, an antimicrobial coating, a thermal-regulating material, and a noise-dampening material to enhance user safety and comfort.

In some embodiments, the supporting frame further incorporates a ventilation unit having integrated fans to maintain breathability and thermal regulation within the containment region. The sidewalls are detachable from the base for cleaning. The multi-functional support system may also comprise a rotational drive mechanism that is configured to rotate the supporting frame at controlled speeds to provide therapeutic effects.

In alternate embodiments, the multi-functional floating apparatus further comprises a removable top barrier enclosing the containment region, and at least one inflatable airbag disposed within the supporting frame. The at least one inflatable airbag is operable via a wearable remote control to selectively adjust buoyancy for the user. The multi-functional floating apparatus further comprises a protective element that is configured to prevent the plurality of weighted modules from contacting a user's head.

The adaptability of the multi-functional support system supports a wide range of user scenarios. For residential users, the multi-functional support system may serve as an alternative to conventional mattresses by providing a waterbed-like experience without the complexity of fluid-based systems. For clinical or therapeutic settings, the multi-functional support system offers pressure-relieving support that aids circulation and rehabilitation. For children or recreational environments, the multi-functional support system provides a safe, buoyancy-mimicking play surface, and for workplace applications. The multi-functional support system may be configured as an ergonomic seating or resting station to reduce fatigue during prolonged activity.

Accordingly, the proposed multi-functional support system represents a significant advancement in ergonomic and therapeutic support technology, delivering the comfort and health benefits of buoyancy in a modular, versatile, and hygienic land-based system. The multi-functional support system provides practical improvements over waterbeds, flotation tanks, and conventional bedding or seating arrangements, while offering scalability and customization for diverse therapeutic, residential, and recreational applications.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a perspective view of a multi-functional support system, in accordance with embodiments of the invention.

FIG. 2 illustrates a perspective view of at least one of plurality of weighted modules, in accordance with embodiments of the invention.

FIG. 3A illustrates a perspective view of an exemplary embodiment, depicting interconnection of the weighted modules, in accordance with embodiments of the invention.

FIG. 3B illustrates a perspective view of another exemplary embodiment, depicting the interconnection of the weighted modules, in accordance with embodiments of the invention.

FIG. 4A and FIG. 4B illustrate perspective views of alternative embodiments of the weighted modules, in accordance with embodiments of the invention.

FIG. 5 illustrates to a perspective view of a first exemplary embodiment of the support system, in accordance with embodiments of the invention.

FIG. 6 illustrates to a perspective view of a second exemplary embodiment of the support system, in accordance with embodiments of the invention.

FIG. 7 illustrates to a perspective view of a third exemplary embodiment of the support system, in accordance with embodiments of the invention.

FIG. 8 illustrates to a perspective view of a fourth exemplary embodiment of the support system, in accordance with embodiments of the invention.

FIG. 9 illustrates to a perspective view of a user with a protective element, in accordance with embodiments of the invention.

FIG. 10 illustrates to a perspective view of the support system with a user positioned within the containment region, in accordance with embodiments of the invention.

FIG. 11A and FIG. 11B illustrate perspective views of the multi-functional support system implemented without a supporting frame, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

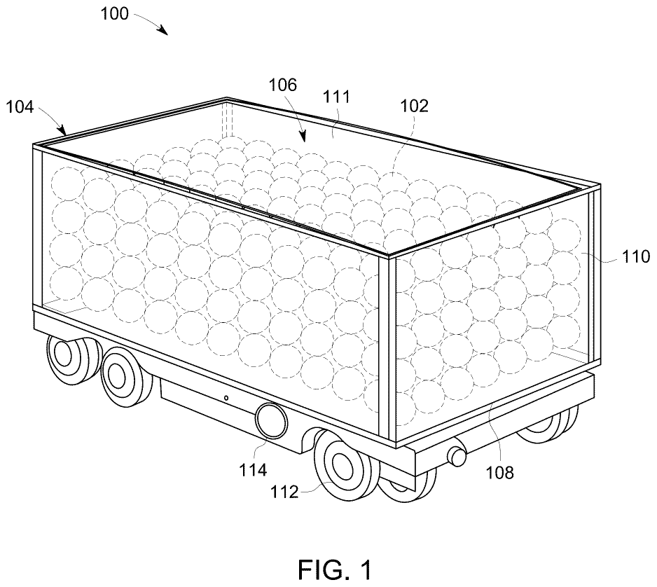

FIG. 1 refers to a perspective view of a multi-functional support system 100. In one embodiment herein, the support system 100 is designed to simulate buoyancy and provide water-like support on a terrestrial surface. The support system 100 achieves this effect by redistributing a user's body weight across a plurality of weighted or buoyancy-regulating modules 102. This redistribution reduces localized pressure points, enhances blood circulation, and improves overall comfort compared to conventional flat surfaces. The support system 100 comprises a supporting frame 104 that defines a containment region 106. In the illustrated embodiment, the supporting frame 104 is rectangular; however, alternative embodiments may employ circular, polygonal, or other geometries to accommodate different applications.

In one embodiment herein, the supporting frame 104 includes a base 108 and sidewalls 110. The sidewalls 110 may be detachably connected to the base 108 using fasteners, interlocking joints, or quick-release mechanisms, thereby facilitating cleaning, drainage, and maintenance of the containment region 106. In some embodiments, the sidewalls 110 incorporate resilient components, such as springs or elastic mounts, to enable lateral displacement of the weighted modules 102 within the containment region 106. This controlled displacement enhances dynamic redistribution of the weighted modules 102 and contributes to a more realistic simulation of floating in a fluid medium.

In one embodiment herein, the support system 100 may incorporate a removable top barrier 111 on top of the supporting frame 104 to regulate airflow and maintain safety. The removable top barrier 111 of the supporting frame 104 may be formed as solid glass or plastic panels, or as metal fences configured with perforations. The removable top barrier 111 restricts direct air currents from external fans or air-conditioning units while maintaining breathable airflow within the containment region 106.

In one embodiment herein, the multi-functional support system 100 is configured for mobility to facilitate use in residential, clinical, and commercial environments. The supporting frame 104 may be mounted on rolling supports 112, such as caster wheels, which permit repositioning of the support system 100 without disassembly. Each caster wheel may include an integrated locking mechanism to secure the support system 100 in place during use. In alternative embodiments, the sidewalls 110 of the supporting frame 104 may be foldable or collapsible, thereby allowing the support system 100 to be compacted for transport or storage. For larger-scale installations, the supporting frame 104 may further comprise at least one of, but not limited to, lifting handles, modular frame segments, and integrated motorized drives to assist in relocation. The incorporation of mobility features enables the support system 100 to be easily cleaned, repositioned, or deployed in multipurpose spaces, thereby enhancing practicality across domestic, therapeutic, and institutional settings.

In one embodiment herein, the weighted modules 102 are dimensioned to define interstitial gaps between adjacent modules. These gaps permit passive air circulation around the user's body, thereby maintaining respiration and promoting thermal comfort during use. To further enhance airflow, the supporting frame 104 may incorporate a ventilation unit 114 configured to maintain oxygen concentration above 19% by volume and to regulate the temperature within the containment region 106. The ventilation unit 114 may include at least one of, but not limited to, perforated sidewalls, integrated fans, and passive airflow channels formed within the supporting frame 104.

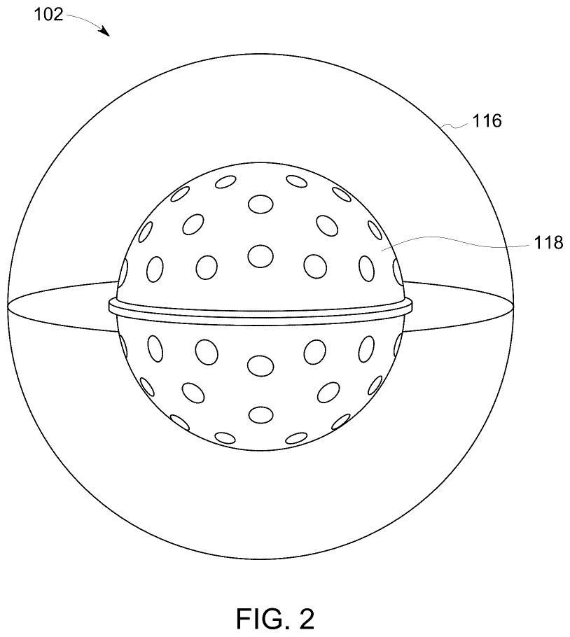

FIG. 2 refers to a perspective view of at least one of the plurality of weighted modules 102. In one embodiment herein, the weighted modules 102 are positioned within the containment region 106 of the support system 100, as depicted in FIG. 1. The plurality of weighted modules 102 is movable relative to the adjacent weighted modules 102 within the containment region 106 by a displacement under a static load, thereby redistributing beneath a user's body to provide buoyant support and reduce localized pressure points. Each weighted module 102 comprises an outer shell 116 enclosing an inner core 118. In one embodiment herein, the inner core 118 comprises a metallic material. In other embodiments, the inner core 118 may comprise at least one of, but not limited to, a liquid, such as saline, and a magnetic material. The materials and composition of the inner core 118 are selected to provide each weighted module 102 with an overall density within approximately ±20% of the average human body density (about 1.0 g/cm³). By maintaining this density balance, the weighted modules 102 collectively support the user in a semi-suspended state, thereby generating a sensation analogous to flotation in water.

In one embodiment herein, the outer shell 116 is spherical in shape. In other embodiments, the outer shell 116 may be at least one of, but not limited to, ellipsoidal and polyhedral. The outer shell 116 may be fabricated from at least one of, but not limited to, transparent polymers, semi-transparent composites, and other durable materials selected for resistance to wear and deformation. The outer shell 116 may further be provided with functional surface coatings, including at least one of, but not limited to, antimicrobial layers to inhibit microbial growth, thermal-regulating layers to stabilize temperature, and noise-dampening finishes to reduce acoustic output during use. In some embodiments, the outer shell 116 may incorporate at least one of, but not limited to, perforations and surface texturing to optimize weight distribution of the inner core 118 and to modulate frictional interaction between the outer shell 116 and the inner core 118. Additionally, the outer shell 116 may be overlaid with at least one of, but not limited to, fuzz-like coatings and rubberized surfaces to further minimize noise and to enhance skin comfort for the user.

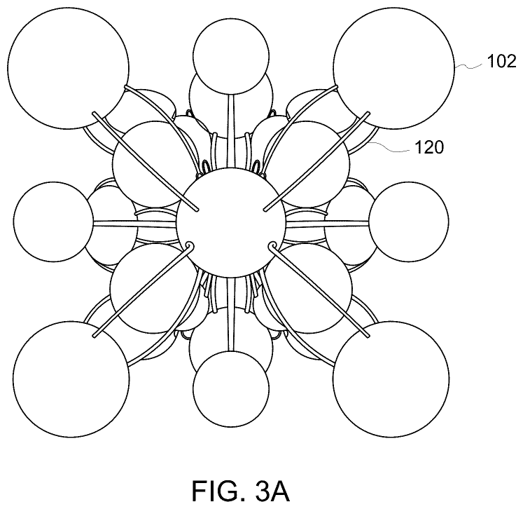

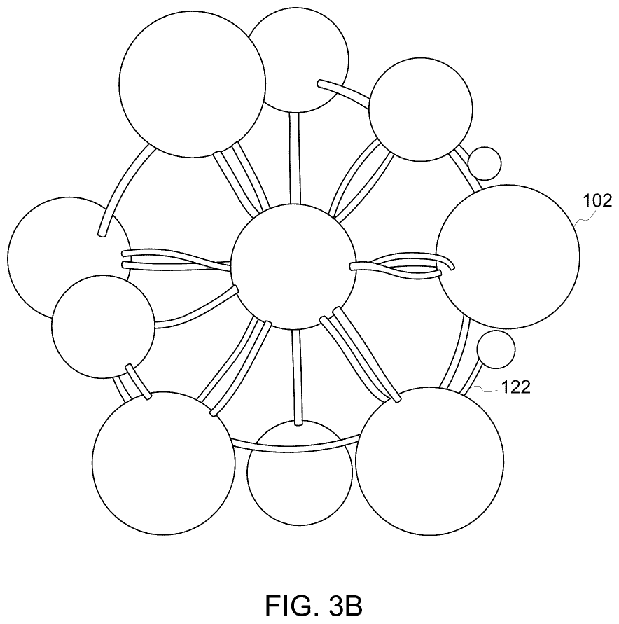

FIG. 3A refers to a perspective view of an exemplary embodiment, depicting interconnection of the weighted modules 102. In this embodiment, the weighted modules 102 are interconnected by elastic members 120 extending between adjacent modules. The elastic members 120 may be formed from resilient materials including at least one of, but not limited to, polymer-coated cables, elastomeric cords, and spring-like rods. These elastic members 120 permit limited displacement of the weighted modules 102 under user load and restore the weighted modules 102 to an equilibrium position when the load is removed. This configuration enhances overall stability of the containment region 106 while preserving controlled flexibility, thereby enabling the user to experience dynamic resistance similar to swimming motions in water.

FIG. 3B refers to a perspective view of another exemplary embodiment, depicting the interconnection of the weighted modules 102. In this embodiment, the weighted modules 102 are joined through flexible linkage elements 122 arranged in a radial or mesh-like configuration. The flexible linkage elements 122 may be constructed from at least one of, but not limited to, metallic rods, reinforced polymer bands, and hybrid composites. The linkage system accommodates both translational and angular movement of each weighted module 102 relative to its adjacent modules. This arrangement maintains uniform spacing, prevents excessive separation or clustering, and ensures continuous body support across the containment region 106. The flexible linkage elements 122 further enhance the simulation of buoyant suspension by permitting coordinated shifting of multiple modules in response to user motion.

Both embodiments (FIGS. 3A and 3B) demonstrate alternative interconnection strategies for the weighted modules 102. The degree of freedom of each weighted module 102 can be tuned by selecting the stiffness of the elastic members 120 in the embodiment of FIG. 3A or by adjusting the compliance of the flexible linkage elements 122 in the embodiment of FIG. 3B. Such tunability allows the support system 100 to be adapted for diverse applications, including high-resistance therapeutic training, low-resistance relaxation, and recreational play environments.

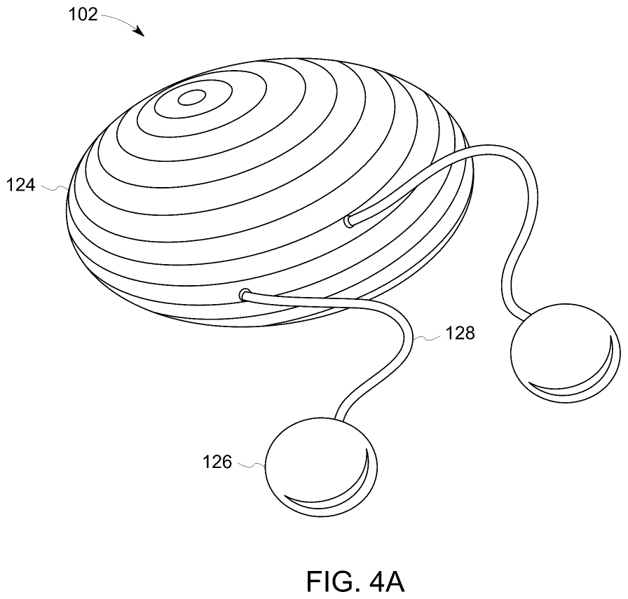

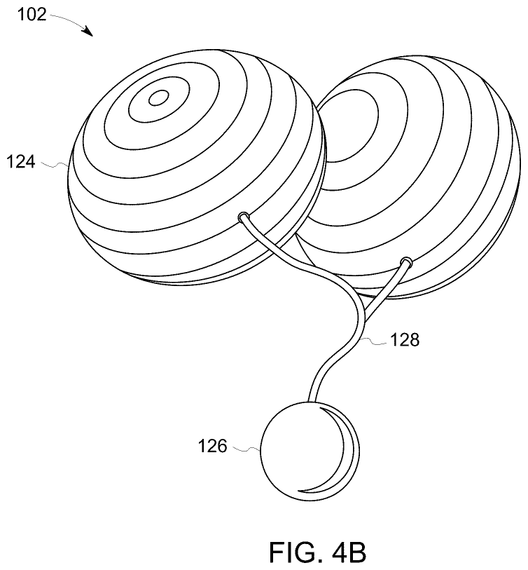

FIG. 4A and FIG. 4B refer to perspective views of alternative embodiments of the weighted modules 102. In these embodiments, the weighted modules 102 may be arranged in composite configurations that combine air-filled modules 124 with metal-core modules 126. In one example, the air-filled module 124 may be elastically connected to one or more metal-core modules 126 by connecting elements 128 include at least one of, but not limited to, bungee cords, elastic bands, and other resilient connectors, as shown in FIG. 4A. In another example, the metal-core module 126 may be interconnected with two or more air-filled modules 124 to form a hybrid assembly, as depicted in FIG. 4B. These composite groupings allow buoyancy characteristics to be finely adjusted by balancing the lighter air-filled modules 124 with the heavier metal-core modules 126. By tailoring the density of the support system 100 to approximate, slightly exceed, or slightly undercut the density of the human body, the support system 100 produces a controlled simulation of buoyant suspension. This results in sensations analogous to swimming or floating in water while maintaining a stable and breathable terrestrial environment.

In another embodiment, the weighted modules 102 may be interconnected by magnetic couplings. Each weighted module 102 may include at least one of, but not limited to, a ferromagnetic insert and the inner core 114 formed of the magnetic material. The adjacent modules are arranged such that magnetic attraction or repulsion forces maintain uniform spacing while permitting limited relative displacement. The magnetic forces may be configured within a range sufficient to prevent uncontrolled clustering of the weighted modules 102 while still allowing smooth redistribution in response to user movement. This embodiment ensures stability of the support surface while preserving the floating sensation, without requiring physical connectors such as cords or linkages.

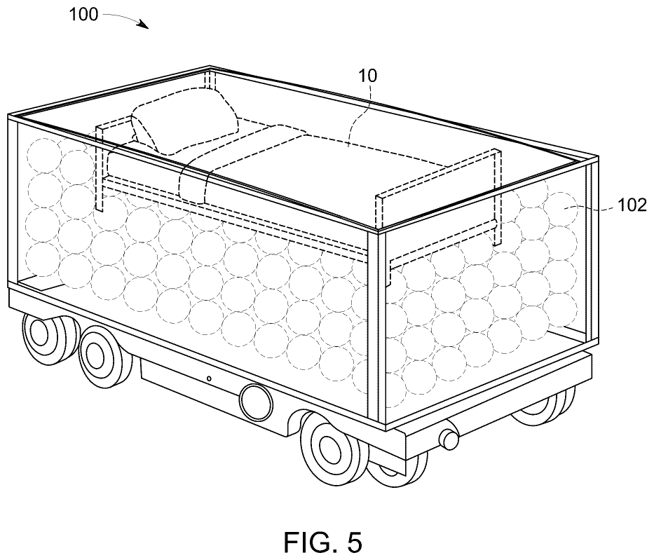

FIG. 5 refers to a perspective view of a first exemplary embodiment of the support system 100. In this embodiment, the support system 100 is configured as a merged floater body bed. A conventional bed structure 10 is positioned above the support system 100, allowing the user to select between two modes of use such as resting directly on the weighted modules 102 to experience buoyant support, or reclining on the conventional bed structure 10 for traditional bedding comfort. The conventional bed structure 10 may be mounted on at least one of, but not limited to, sliding, hinged, and retractable mechanisms configured to permit lateral, vertical, or equivalent movement. This design enables convenient transition between the support system 100 and the conventional bed structure 10. The merged configuration thus provides versatility for diverse user preferences and enhances accessibility for individuals seeking either therapeutic flotation or standard rest.

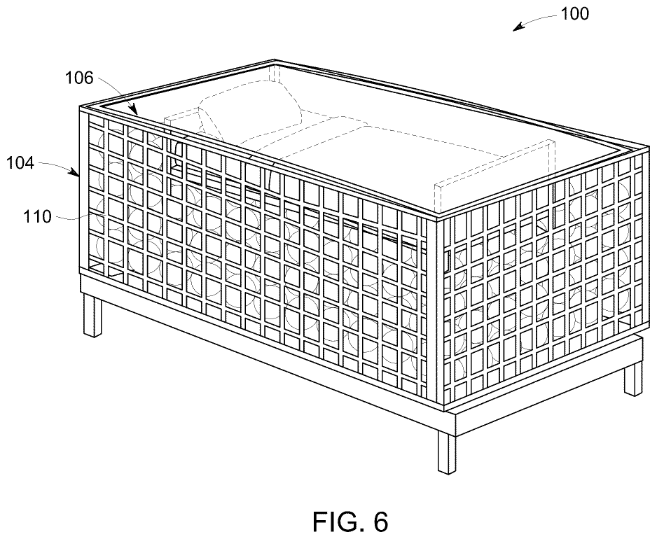

FIG. 6 refers to a perspective view of a second exemplary embodiment of the support system 100. In this embodiment, the sidewalls 110 of the supporting frame 104 may be constructed from materials other than glass or plastic. For example, the sidewalls 110 may be formed from a material including at least one of, but not limited to, a metal fence, a perforated glass and a plastic wall. These configurations allow ambient air circulation into the containment region 106 while preventing direct airflow from fans or air-conditioning units positioned below the support system 100. Such arrangements maintain adequate oxygen levels within the containment region 106 and enhance user comfort without requiring additional ventilation equipment.

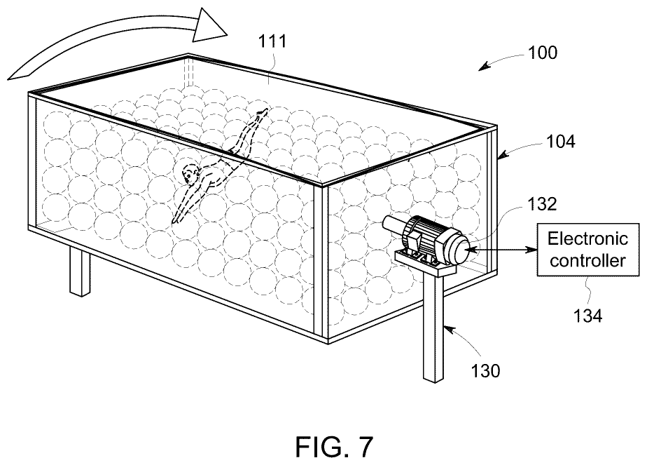

FIG. 7 refers to a perspective view of a third exemplary embodiment of the support system 100. In this embodiment, the support system 100 may be configured as a rotating body floater. The supporting frame 104 may be mounted on a rotational drive mechanism 130 that enables the containment region 106 and the plurality of weighted modules 102 to rotate at a controlled speed. The rotational speed may be selected to avoid discomfort or dizziness, and may range from approximately one revolution per minute to one revolution per hour. This controlled rotation provides vestibular stimulation that can promote improved circulation, enhanced relaxation, and cognitive benefits. In this embodiment, the removable top barrier 111 may further serve to retain the weighted modules 102 and to preserve internal air pressure, thereby ensuring consistent buoyant support during motion.

In this embodiment, the user may recline directly on the weighted modules 102 while the support system 100 is in motion. The collective redistribution of the weighted modules 102 during rotation maintains uniform body support and permits limited swimming-like movement. The buoyant sensation combined with the rotational motion creates a unique therapeutic and recreational experience that differs from static flotation systems. The rotational drive mechanism 130 may be implemented using a motorized bearing assembly 132, with speed regulated by electronic controllers 134. The integration of flotation with controlled rotation provides a versatile platform that may be adapted for wellness, therapeutic, or recreational applications.

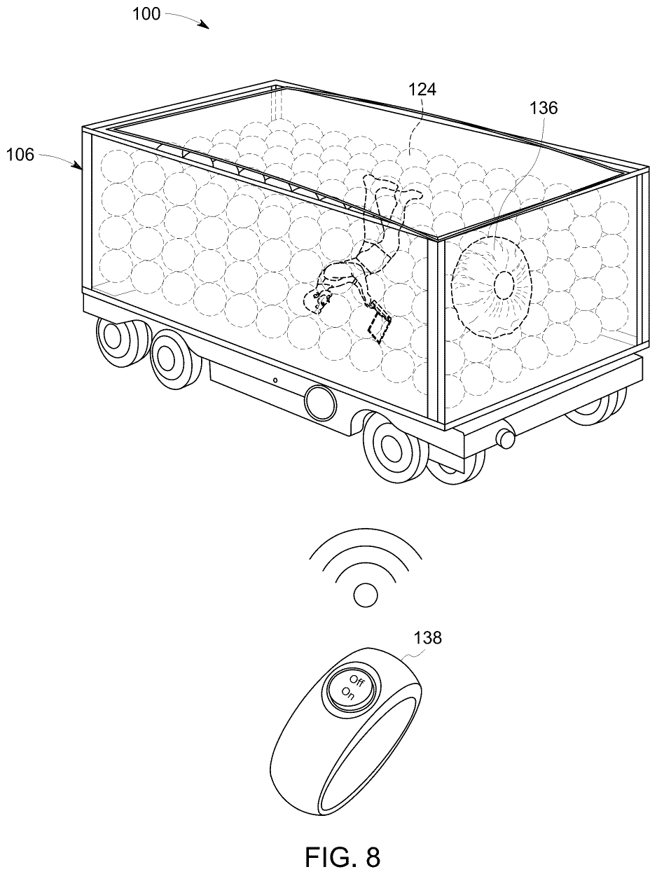

FIG. 8 refers to a perspective view of a fourth exemplary embodiment of the support system 100. In this embodiment, the support system 100 may be configured with lightweight air-filled modules 124 that contain no liquid or metallic cores, but instead are fully inflated with air. To enhance buoyant support in this configuration, an inflatable airbag 136 may be positioned within the containment region 106. The inflatable airbag 136 may be inflated to a level sufficient to maintain the user in a semi-suspended state, thereby preventing the body from sinking to the base 108 of the support system 100. The inflatable airbag 136 may be operable by a wearable remote control 138, allowing the user to adjust inflation in real time for comfort and buoyancy control. For safety, the inflatable airbag 136 may be designed with pressure limits to prevent over-inflation that could restrict movement. In some embodiments, a secondary backup airbag with an independent remote control may be provided to ensure redundancy in the event of failure of the inflatable airbag 136. This air-filled configuration offers a simplified version of the support system 100, while remaining compatible with other embodiments, including rotational or spinning configurations.

In yet another embodiment, the supporting frame 104 may incorporate drainage ports and sanitizable surfaces to facilitate cleaning and hygiene. The weighted modules 102 may be provided with antimicrobial surface coatings to inhibit bacterial growth, while the smooth outer shells 112 are configured to minimize debris accumulation. In some embodiments, the support system 100 may further include automated spraying systems and self-cleaning surface treatments to enable rapid sanitation and reduce maintenance requirements.

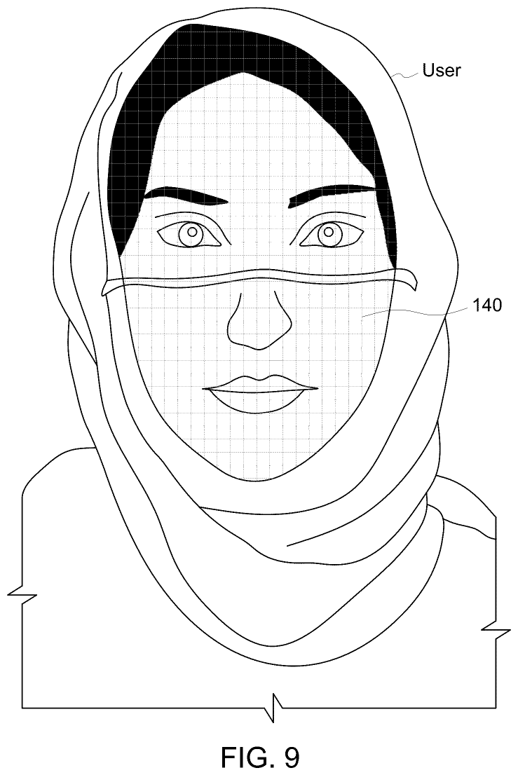

FIG. 9 refers to a perspective view of the user with a protective element 140. In one embodiment herein, the weighted modules 102 (shown in FIG. 1) may be manufactured in varying sizes to achieve different functional effects. Smaller modules facilitate finer redistribution of load and smoother motion of the user's limbs, thereby enhancing comfort and responsiveness. Larger modules provide increased structural support and stability. In all configurations, the diameter of each weighted module 102 is selected to be greater than the average dimensions of human nasal and auditory passages, thereby preventing accidental entry into those passages. In embodiments utilizing the smaller modules, additional safety measures may be incorporated, such as the protective element 140 that is either positioned adjacent to the user's head or configured to be worn by the user, thereby further reducing the risk of inadvertent contact or entry.

In another embodiment, the weighted modules 102 may be configured as lightweight magnetic hollow balls. Instead of incorporating liquid or metallic cores to achieve body-equivalent density, the outer shell 116 of each weighted module 102 may include a thin magnetic layer. The magnetic strength of the weighted modules 102 is selected to be sufficient to prevent the user's body from sinking fully to the base 108 of the support system 100, while not so strong as to prevent partial immersion into the array of modules. This configuration provides a buoyant sensation similar to the weighted modules 102 containing metal or liquid cores, while reducing the overall weight of the support system 100 to facilitate portability and transportation.

In an alternative embodiment, elasticity-based suspension may be employed instead of magnetic forces. In this configuration, each weighted module 102 is connected to the base 108 of the supporting frame 104 by the elastic members 120 such as bungee cords. The elastic members 120 may be arranged vertically such that each row of the weighted modules 102 is supported at progressively varying cord lengths, with shorter cords near the upper rows and longer cords near the lower rows. This arrangement permits controlled lateral displacement of the weighted modules 102 when the user enters the support system 100. As the user presses downward, the upper weighted modules 102 shift slightly to the side while being restrained by the cords, and subsequent lower rows respond in a similar manner. This sequential redistribution creates a layered resistance effect that simulates the sensation of swimming or floating in water.

In a preferred embodiment, the multi-functional support system 100, also referred to as a multi-functional floating apparatus, is constructed as a rectangular unit measuring approximately 2.0 m in length, 1.5 m in width, and 0.8 m in height, suitable for accommodating an adult user. The supporting frame 104 may be fabricated from reinforced polymer with the sidewalls 110 secured to the base 108 by interlocking joints and quick-release fasteners. The sidewalls 110 may incorporate integrated springs to facilitate lateral sliding of the weighted modules 102 during use. Drainage ports are formed in the base 108 to permit removal of fluids during cleaning.

The containment region 106 houses the weighted modules 102 arranged in a grid-like pattern. Each weighted module 102 comprises the transparent polycarbonate outer shell 116 of approximately 100 mm to 150 mm in diameter. The metallic inner core 118 enclosed within each outer shell 116 is suspended in a saline solution, thereby providing an overall module density of approximately 1.0 g/cm³, corresponding to human body density. This configuration ensures that when the user rests on the weighted modules 102, body weight is evenly redistributed and the user experiences a buoyant sensation similar to water flotation.

The outer shells 116 of the weighted modules 102 are coated with an antimicrobial layer formulated to reduce bacterial growth by at least 90% compared to uncoated polymers. In addition, a thin rubberized surface layer provides noise dampening, thereby reducing module-to-module contact sounds to less than 40 dBA at a distance of 0.5 m. The weighted modules 102 are dimensioned such that their minimum diameter exceeds the average dimensions of human nasal and auditory passages, thereby ensuring user safety. For additional protection, the protective element 140 may be mounted near the user's head to prevent accidental contact with the weighted modules 102 when smaller-sized versions are used. The ventilation unit 116 is integrated into the supporting frame 104 to maintain airflow. The support system 100 includes perforated side panels and low-noise fans configured to maintain oxygen concentration above 19% by volume inside the containment region 106, while also regulating internal temperature within ±2° C. of ambient. This ensures continuous breathability for the user during prolonged sessions.

The weighted modules 102 are interconnected by the elastic members 120, allowing controlled compression and resistance as the user shifts to enhance functionality. This provides a sensation akin to swimming in a fluid medium. For advanced therapeutic applications, the supporting frame 104 may be coupled to the rotational drive mechanism 130 capable of rotating the containment region 106 at a controlled speed of 0.1 to 2 revolutions per minute. This controlled rotation induces vestibular stimulation, improving relaxation, circulation, and neurological benefits. The inflatable airbags 136 may be positioned within the supporting frame 104 to stabilize the user during rotational operation while still permitting limited motion.

Hygiene and maintenance are facilitated by the sidewalls 110 and smooth sanitizable surfaces. The support system 100 is mounted on the rolling supports 112, enabling transport to a cleaning station. Automated spray nozzles may be integrated for rapid washing and drainage. The antimicrobial coatings on the weighted modules 102 further reduce bacterial growth between cleaning cycles, making the support system 100 suitable for repeated use in residential, clinical, and commercial environments. In operation, the user reclines onto the plurality of weighted modules 102. The weighted modules 102 redistribute under the user's body, conforming to body contours and reducing localized pressure points by at least 30% compared to a flat mattress. The combined action of module density, elastic interconnections, and ventilation produces a stable, breathable, and comfortable environment emulating buoyancy in water, without the risks or complexities associated with water-based flotation systems.

The multi-functional support system 100, herein referred to as the multi-functional floating apparatus, is designed to accommodate diverse applications across residential, therapeutic, recreational, and occupational environments. In residential settings, the support system 100 may function as a bed or relaxation platform. Traditional mattresses concentrate body weight at pressure points, thereby leading to discomfort and circulatory restriction. In contrast, the support system 100 redistributes weight evenly across the weighted modules 102 to reduce pressure by at least 30% compared to conventional bedding. The floating sensation allows users to experience water-like buoyancy without the disadvantages of waterbeds, such as leakage, instability, or complex maintenance. Optional integrated ventilation ensures breathability for extended overnight use, while antimicrobial coatings maintain hygiene in shared environments.

In clinical and rehabilitative environments, the support system 100 may be employed to alleviate musculoskeletal stress and promote patient recovery. The semi-suspended state achieved by the weighted modules 102 reduces gravitational loading on joints and muscles, thereby facilitating relaxation and faster healing. The rotational drive mechanism 130 may be utilized to stimulate vestibular systems, thereby supporting therapy for neurological conditions such as balance disorders. Elastic and magnetic interconnections between the weighted modules 102 further enhance the sensation of controlled resistance, thereby replicating gentle aquatic exercise for patients unable to access water-based therapy.

In pediatric applications, the support system 100 may be configured as a safe play environment. The containment region 106 may be dimensioned with reduced depth, while protective mesh and oversized modules prevent accidental ingestion or choking hazards. Children benefit from tactile exploration of the weighted modules 102, which move responsively to body motion, thereby fostering motor skill development and sensory engagement. Unlike swimming pools, which carry inherent drowning risks, the support system 100 provides buoyant play without submersion in liquid, thereby allowing for safer recreational experiences. Optional colored, textured, or illuminated weighted modules 102 may further enhance play value.

In office or workspace settings, the support system 100 may serve as an ergonomic body floater for prolonged sitting or reclining. Modern work environments often require extended periods of sedentary posture, thereby leading to discomfort and decreased productivity. The buoyant support provided by the weighted modules 102 alleviates spinal compression and muscular fatigue. Integration with ventilation and temperature control systems ensures long-duration comfort. For hybrid workspace applications, the supporting frame 104 may be dimensioned to permit laptop or workstation use while the user remains supported on the weighted modules 102.

The support system 100 may also be deployed in specialized environments such as hotels, spas, or wellness centers. For commercial usage, the supporting frame 104 may be equipped with the rolling supports 112 and the sidewalls 110 for rapid sanitation between the users. Automated cleaning stations with integrated spray nozzles may be configured to disinfect the weighted modules 102 and drain residual water. In premium environments, the containment region 106 may be scaled to room size, allowing multiple occupants to share buoyant experiences similar to communal pools, while maintaining breathability and comfort not achievable in traditional water-based flotation.

Across all use cases, the support system 100 incorporates safety features such as the protective element 140 to the user's head when smaller weighted modules 102 are used, ingress and egress aids such as steps, ramps, or handrails, redundant inflatable airbags 136 with independent controllers 134 for buoyancy adjustment, antimicrobial coatings to suppress bacterial growth, and smooth sanitizable surfaces for hygienic maintenance. These features ensure compliance with medical, residential, and commercial safety standards, thereby extending the support system's 100 applicability across multiple sectors.

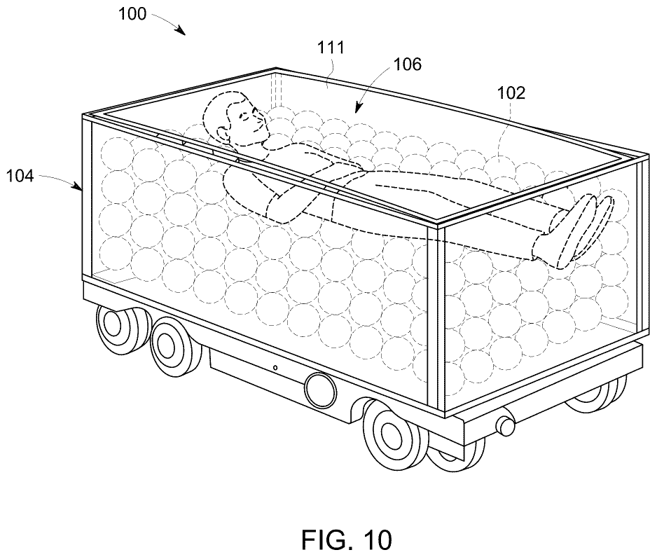

FIG. 10 refers to a perspective view of the support system 100 with the user positioned within the containment region 106. The user reclines on the plurality of weighted modules 102, which redistribute under the user's body to provide uniform buoyant support. The weighted modules 102 collectively conform to the contours of the user, thereby reducing localized pressure points and simulating the sensation of floating in water. The supporting frame 104 defines the containment region 106 and retains the weighted modules 102, thereby ensuring stability during use. This configuration demonstrates the functional interaction between the user and the support system 100 in providing flotation-like comfort on a terrestrial surface.

In another embodiment, the weighted modules 102 may be configured as magnetic repulsion modules. Each weighted module 102 may include the inner core 114 formed of magnetic material arranged such that adjacent weighted modules 102 repel one another. The repulsive force maintains uniform spacing between the weighted modules 102 and provides a distributed support surface without requiring physical connectors. The magnetic repulsion strength may be tuned to allow the user to sink partially into the array under gravity, reaching a natural equilibrium similar to floating in water. In some embodiments, the outer shell 112 of the weighted modules 102 may be fabricated from smooth polymers or coated with thin plastic layers to improve safety, reduce friction, and enhance comfort.

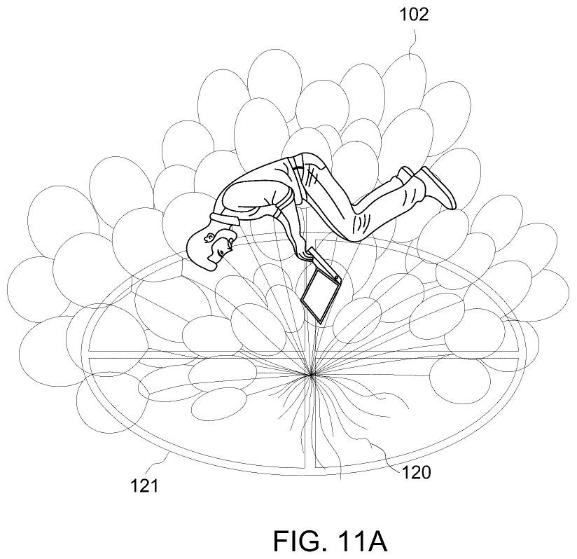

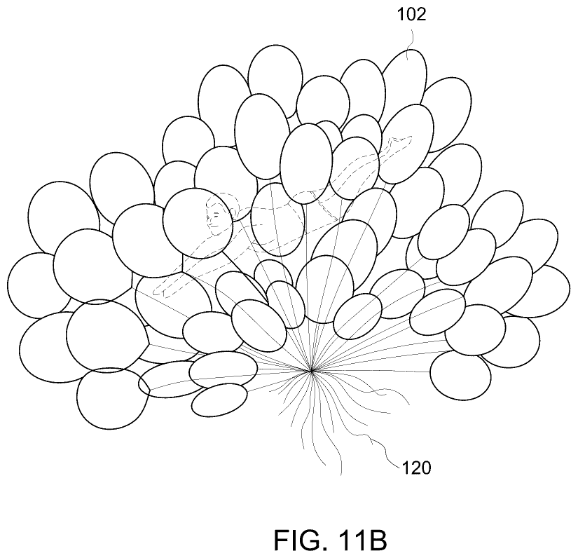

FIG. 11A and FIG. 11B refer to perspective views of the multi-functional support system 100 implemented without a supporting frame 104. In these configurations, a plurality of air-filled modules 124 may be interconnected by elastic members 120, such as bungee cords, converging toward one or more fixed points or bases. The air-filled modules 124 collectively form a free-standing buoyant cluster into which the user may recline or dive. As shown in FIG. 11A, the elastic members 120 may be anchored to a rigid base 121, which may be metallic or composite, to maintain structural integrity and preserve cord strength during repeated use. This arrangement enables buoyant support without requiring a rigid containment frame, thereby reducing weight and simplifying portability.

The present invention provides the multi-functional support system 100 that replicates buoyant support without requiring a water medium. By employing the weighted modules 102 configured with densities approximating that of the human body, the support system 100 redistributes body weight to reduce localized pressure points, enhance blood circulation, and improve musculoskeletal comfort. The modular structure enables conformal support across the body, thereby providing sensations similar to aquatic flotation while avoiding limitations such as water maintenance, hygiene concerns, and immersion risks. Additional embodiments incorporating the elastic members 120, the flexible linkages 122, or magnetic couplings allow controlled displacement of weighted modules 102 to enhance responsiveness and stability. Integration of the ventilation unit 114 within the supporting frame 104 ensures breathability, thermal regulation, and oxygen circulation, thereby overcoming limitations of enclosed flotation systems. Further, optional rotational drive mechanisms 130 provide vestibular stimulation, thereby contributing to therapeutic, neurological, and relaxation benefits.

From a commercial perspective, the invention offers a versatile and scalable platform that can be adapted for residential, therapeutic, recreational, and institutional environments. Unlike water-based flotation tanks or hydrotherapy pools, the support system 100 eliminates the need for large volumes of water, plumbing infrastructure, and continuous maintenance, thereby significantly reducing operational costs. The modular design permits straightforward cleaning through antimicrobial coatings, detachable sidewalls, drainage ports, and automated sanitation systems, making it suitable for high-traffic facilities such as hospitals, wellness centers, and spas. The mobility features, such as the roller supports 112, enable easy repositioning, thereby increasing flexibility in multipurpose spaces. Additionally, the support system 100 can be customized with hybrid module configurations, merged conventional beds, or lightweight portable embodiments, thereby broadening its commercial applicability across consumer wellness, medical rehabilitation, hospitality, and fitness sectors.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

1. A multi-functional support system for simulating buoyancy, comprising:
a supporting frame defining a containment region; and
a plurality of weighted modules disposed within the containment region, wherein each of the plurality of weighted modules comprises an outer shell enclosing an inner core having a density to approximate human body density,
wherein the plurality of weighted modules is movable relative to the adjacent weighted modules within the containment region by a displacement under a static load, thereby redistributing beneath a user's body to provide buoyant support and reduce localized pressure points.

2. The multi-functional support system of claim 1, wherein the plurality of weighted modules is interconnected by at least one of elastic members, magnetic couplings, and flexible linkages.

3. The multi-functional support system of claim 1, wherein the inner core comprises at least one of a liquid, a metallic material, and a magnetic material.

4. The multi-functional support system of claim 1, wherein the outer shell is formed of a material includes at least one of a transparent polymer and a semi-transparent composite material.

5. The multi-functional support system of claim 1, wherein the outer shell comprises a shape includes at least one of a spherical, an ellipsoidal, and a polyhedral.

6. The multi-functional support system of claim 1, wherein the inner core comprises a surface texturing and perforations to enhance weight distribution and friction within the outer shell.

7. The multi-functional support system of claim 1, wherein the outer shell is coated with a material includes at least one of an antimicrobial material, a thermal-regulating material, and a noise-dampening material.

8. The multi-functional support system of claim 1, wherein the supporting frame comprises a base, sidewalls that are detachable from the base to facilitate cleaning and drainage, and a removable top barrier configured to enclose the containment region.

9. The multi-functional support system of claim 1, wherein the multi-functional support system further comprises at least one protective element selected from at least one of netting, guards, and mesh structures, wherein the protective element is configured to prevent the plurality of weighted modules from entering the user's nasal and auditory passages.

10. The multi-functional support system of claim 1, wherein the multi-functional support system further comprises a ventilation unit integrated into the supporting frame to enhance breathability within the containment region.

11. The multi-functional support system of claim 1, wherein the supporting frame further comprises rolling supports with locking mechanisms to enable repositioning.

12. The multi-functional support system of claim 1, wherein the multi-functional support system further comprises at least one inflatable airbag disposed within the supporting frame, wherein the at least one inflatable airbag is operable via a wearable remote control to selectively adjust buoyancy for the user.

13. A multi-functional floating apparatus for simulating buoyancy, comprising:
a supporting frame having sidewalls and a base defining a containment region;
a plurality of weighted modules disposed within the containment region, wherein each of the plurality of weighted modules comprises an outer shell enclosing an inner core having a density to approximate human body density,
wherein the plurality of weighted modules is movable relative to the adjacent weighted modules within the containment region by a displacement under a static load, thereby redistributing beneath a user's body to provide buoyant support and reduce localized pressure points;
plurality of elastic members interconnecting the plurality of weighted modules to allow controlled redistribution; and
a ventilation unit integrated into the supporting frame to enhance breathability within the containment region.

14. The multi-functional floating apparatus of claim 13, wherein the outer shell of each of the plurality of weighted modules is coated with an antimicrobial material to inhibit bacterial growth.

15. The multi-functional floating apparatus of claim 13, wherein the outer shell of each of the plurality of weighted modules is coated with a noise-dampening material to reduce sound and enhance comfort.

16. The multi-functional floating apparatus of claim 13, wherein the side walls of the supporting frame are detachable from the base to facilitate cleaning and drainage.

17. The multi-functional floating apparatus of claim 13, wherein the ventilation unit comprises integrated fans configured to regulate oxygen and temperature within the containment region.

18. The multi-functional floating apparatus of claim 13, wherein the multi-functional floating apparatus further comprises a removable top barrier enclosing the containment region, and at least one inflatable airbag disposed within the supporting frame, and wherein the at least one inflatable airbag is operable via a wearable remote control to selectively adjust buoyancy for the user.

19. The multi-functional floating apparatus of claim 13, wherein the multi-functional floating apparatus further comprises a protective element that is configured to prevent the plurality of weighted modules from contacting a user's head.

20. The multi-functional floating apparatus of claim 13, wherein the multi-functional floating apparatus further comprises a rotational drive mechanism to rotate the supporting frame at a controlled speed to provide therapeutic effects.
